(19) United States
(12) Patent Application Publication
(10) Pub. No.: US 2023/0363951 A1
(43) Pub. Date: Nov. 16, 2023

(54) GOGGLE ASSEMBLY

(71) Applicant: Michael Cooper, Red Hill QLD (AU)

(72) Inventor: Michael Cooper, Red Hill QLD (AU)

(21) Appl. No.: 18/197,444

(22) Filed: May 15, 2023

(30) Foreign Application Priority Data
May 16, 2022 (AU) 2022901302

(51) Int. Cl. A61F 9/02

(52) U.S. Cl. CPC A61F 9/028; A61F 9/029

(57) ABSTRACT

The present invention resides in a goggle assembly comprising a frame supporting a lens, wherein the frame and/or lens comprise aperture(s) at an upper end thereof or therebetween, a strap connected to frame and a duct connected to frame and/or lens at a side or bottom thereof, wherein the duct is adapted to vent air to the external environment. The present invention provides an improved goggle assembly for thermoregulation.

### CROSS-REFERENCE TO RELATED APPLICATION

This application claims the benefit of priority under 35 U.S.C. § 119(a) to Australian Patent Application No. 2022901302, filed May 16, 2022, the entire contents of which are incorporated by reference in their entirety.

### FIELD OF THE INVENTION

The present invention relates to the field of eye protection. Particularly, the present invention resides in an improved goggle assembly for sports and other activities More particularly, the invention relates to a goggle assembly which alleviate the problem of inadequate thermoregulation.

### BACKGROUND TO THE INVENTION

Any reference to background art herein is not to be construed as an admission that such art constitutes common general knowledge in Australia or elsewhere.

Eyewear is required for protection of the eyes for numerous sports and activities. In particular, sports that are at speed are particularly susceptible to injury due to matter flying through the air. However, an issue with the presently available protective eyewear (such as goggles) is that they can fog up due to the temperature differential between the eye cavity and the external environment, and moisture buildup. Furthermore, thermoregulation using these goggles is also an issue. Additionally, fogging and overheating may also be a disadvantage to goggles and this can be addressed through addressing the thermoregulation issue.

During motorcycling or other strenuous exercises, it may not be possible to remove the fog from the goggle cavity. In this regard, typically, the user is required to physically adjust the goggles to remove heat and thus moisture from the cavity. It will be appreciated that during strenuous exercise or motorcycling/cycling, it may not be possible to complete this as it can be very dangerous.

This issue is particularly prominent in motorcycling as there can be dirt and sand sprayed up by the person riding in front. Additionally, motorcycling is completed at speed and the user may not be able to free their hands to adjust the goggles. Furthermore, heat stress and fatigue of the user can occur due to heat buildup due to inadequate airflow.

It would be advantageous to address one or more of the above issues and/or to provide a commercial alternative to the consumer.

### SUMMARY OF THE INVENTION

In first aspect, although it need not be the only or indeed the broadest form, the invention resides in a goggle assembly comprising:
a frame supporting a lens, wherein the frame and/or lens comprise aperture(s) at an upper end thereof or therebetween;
a strap connected to frame; and
a duct connected to frame and/or lens at a side or bottom thereof.

In one embodiment, the frame and/or lens comprise the aperture(s) at an upper end thereof. In an embodiment, the aperture is formed between the frame and lens. In one embodiment, the frame comprises the apertures at an upper end thereof. In another embodiment, the lens comprises the apertures at an upper end thereof.

In certain embodiments, the aperture(s) are in the form of a set of apertures. In a further embodiment, the goggle assembly comprises a single set of apertures.

In one embodiment, the goggle assembly further comprises a filter insert. In embodiments, the aperture(s) is or are adapted to receive a filter insert. In certain embodiments, the filter is adapted to filter the air and/or adapted to control fluid flow therethrough

In one embodiment, the duct is adapted to vent air to the external environment. In an embodiment, the duct comprises a passageway. In embodiments, the duct is formed with the strap.

In an embodiment, the google assembly further comprises an adjustable valve. In embodiments, the adjustable valve is adapted to control fluid flow through the duct. In one embodiment, the duct comprises the adjustable valve. In certain embodiments, the adjustable valve is located at an outlet of the duct. In another embodiment, the adjustable valve is located along the duct.

The various features and embodiments of the present invention referred to in the individual sections above and in the description which follows apply, as appropriate, to other sections, Mutatis mutandis. Consequently, features specified in one section may be combined with features specified in other sections as appropriate.

Further features and advantages of the present invention will become apparent from the following detailed description.

### BRIEF DESCRIPTION OF THE DRAWING

To assist in understanding the invention and to enable a person skilled in the art to put the invention into practical effect, embodiments of the invention will be described by way of example only with reference to the accompanying drawing, in which:

FIG. 1 is an embodiment of the present goggle assembly

### DETAILED DESCRIPTION OF THE INVENTION

Embodiments of the present invention reside primarily in a goggle assembly. Accordingly, the assembly has been illustrated in concise schematic form in the drawings, showing only those specific details that are necessary for understanding the embodiments of the present invention, but so as not to obscure the disclosure with excessive detail that will be readily apparent to those of ordinary skill in the art having the benefit of the present description.

In this specification, adjectives such as first and second, left and right, and the like may be used solely to distinguish one element or action from another element or action without necessarily requiring or implying any actual such relationship or order.

Words such as “comprises” or “includes” are intended to define a non-exclusive inclusion, such that a process, method, article, or assembly that comprises a list of elements does not include only those elements but may include other elements not expressly listed, including elements that are inherent to such a process, method, article, or assembly.

As used herein, the term ‘about’ means the amount is nominally the number following the term ‘about’ but the actual amount may vary from this precise number to an unimportant degree.

The present invention is predicated on the finding that the present goggle assembly alleviates the problem of thermoregulation and thus fogging in goggles. In this regard, the present invention allows for active heat removal from the goggle cavity without substantial input by the user.

In first aspect, although it need not be the only or indeed the broadest form, the invention resides in a goggle assembly comprising a frame supporting a lens, wherein the frame and/or lens comprise aperture(s) at an upper end thereof or therebetween, a strap connected to frame; and a duct connected to frame and/or lens at a side of bottom thereof.

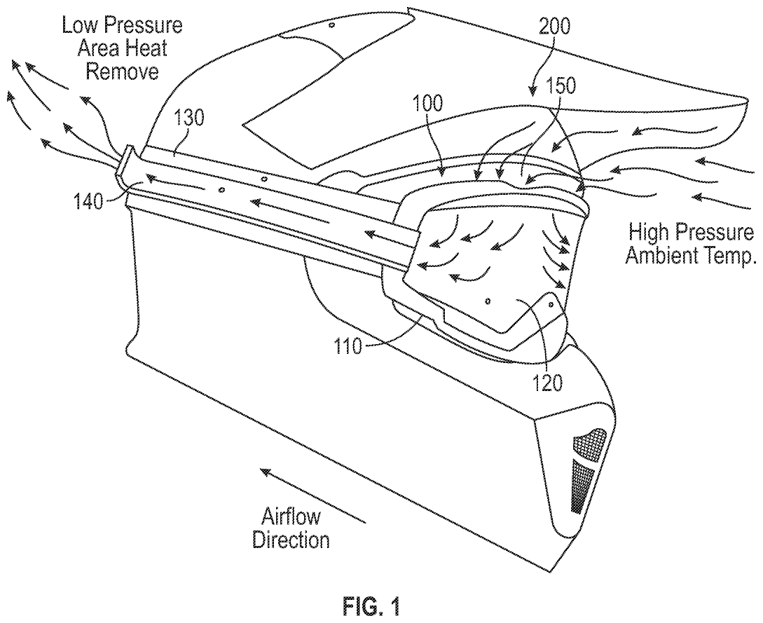

Shown in FIG. 1 is an embodiment of the present goggle assembly 100. The goggle assembly 100 comprises a frame 110. The frame 110 supports a lens 120. The lens 120 is a transparent layer in which the user can see therethrough. The frame 110 is adapted to abut the users face of head (as shown in FIG. 1). The frame 110 may suitably comprise a deformable material that conforms to the user's face. The person skilled in the art will appreciate that this provides the user with comfort whilst wearing the goggles. Furthermore, the person skilled in the art will appreciate that any material can be utilized for this use. As shown, the present goggle assembly may be utilized with a helmet assembly 200.

The goggle assembly 100 further comprises a strap 130 connected to the frame 110. The strap 130 is connected to the frame at opposing ends thereof. The strap 130 holds the frame 110 to the user's face. It will be appreciated the strap is suitably formed of a resilient material. In one embodiment, the strap is an elastic strap. In this regard, the strap is suitably formed of an elastic material. It will be appreciated that, in use, the frame and lens along with the user's face defines a goggle cavity.

The frame 110 and/or lens 120 may comprise aperture(s) 150 at an upper end thereof. The frame 110 and/or lens 120 may suitably comprise a single set of aperture(s) 150 at an upper end of That is, the goggle assembly comprises a single set of aperture(s) at the upper end thereof, and other apertures are either not present or are substantially blocked. In this regard, apertures at the lower end of the frame and/or lens may be blocked to substantially prevent fluid communication between the external environment and the goggle assembly.

The aperture(s) may be considered inlet apertures whereby, in use, airflow generally flow therethrough into the goggle cavity. In one embodiment, the goggle assembly only comprises inlet apertures at an upper end of the frame and/or lens, or between the frame and lens. That is, for completeness, no other inlet apertures are present in the goggles.

The apertures(s) 150 provide fluid communication between the goggle cavity and the external environment. The aperture(s) 150 allow for air from the external environment to enter the goggle cavity (discussed in more detail hereinafter). In one embodiment, the aperture(s) 150 may be formed at the junction between the frame 110 and/or lens 120. In this regard, the aperture(s) 150 may be in the form of a void or gap between the frame and lens. The aperture(s) 150 are suitably formed at the upper end of the goggle assembly. That is, the aperture(s) 150 are suitably formed at the upper end of the frame 110 and/or, or between, the lens 120. In an embodiment, the aperture(s) 150 is/are adapted to receive a filter insert. In one embodiment, the aperture(s) 150 are provided with a filter to remove dirt, dust or particulate matter from the air entering the goggle cavity.

The filter insert may also be utilized to control the fluid flow through the aperture. Furthermore, the filter insert can be used to control the pressure in the goggle cavity which can reduce chance of debris ingress and control the amount of heat load removal required depending on the ambient conditions. The person skilled in the art will appreciate that a number of different tools or techniques can be utilized to control the flow of fluid into and through the goggle cavity. This allows for the control of the amount of heat load removal. This is particularly useful depending on the conditions (mentioned in more detail hereafter).

The goggle assembly 110 suitably comprises a duct 140. The duct 140 suitably provides a passageway between the goggle cavity and the external environment. The duct 140 is suitably in fluid communication with the external environment and the goggle cavity. The duct 140 is adapted to vent air in the google cavity to the external environment. In one embodiment, the duct 140 comprises an elongate passageway that extends from the bottom or sides of the goggle assembly such that a passageway is provided towards the rear of the goggle assembly.

In one embodiment, the duct 140 is preferably formed with, and/or adjacent, the strap 130. In this embodiment, the duct is not compressed and this allows an uninterrupted passageway for air in the google cavity. In one embodiment, the passageway comprises an elongate tube or pipe. It is postulated that the elastic nature of the strap prevents collapse of the duct. In one embodiment, the duct 140 is formed of a resilient material. A non-limiting example of the material forming the duct is silicone.

The goggle assembly 100 further comprises one or more filter inserts. The filter inserts are suitably adapted to restrict the airflow at the duct. In an embodiment, the insert comprises a perforated medium. In one embodiment, the perforated medium is an open cell foam. In an embodiment, the perforated medium has a predetermined micron rating that allows the desired flow control. It will be appreciated that essentially any filter insert with any micron rating can be utilized with the present invention. In this regard, the amount of airflow restricted by the filter can be chosen based on the environment in which the goggle assembly is utilized.

In another embodiment, the goggle assembly 100 further comprises an adjustable valve. In one embodiment, the adjustable valve comprises a dial adapted to control the flow of air through the duct. The adjustable valve is suitably located at an outlet of the duct 140, or along the duct 140. The valve can be adjusted such that the amount of air flow provided by the duct can be controlled. This allows for the user to control the amount of flow. The optimal setting can be chosen for the conditions. In this regard, the settings can be adjusted for the conditions (e.g., high speed or low speed). In one embodiment, the valve is a needle valve.

The person skilled in the art will appreciate there are multiple techniques that can be utilized to control the amount of fluid flow. In this regard, the use of different porosity filter material in the filter insert can be utilized to control the fluid flow. Alternatively, or additionally, the use of an adjustable valve may be utilized to control the fluid flow. In one embodiment, the goggle assembly utilizes the filter insert and/or the adjustable vale. Alternatively, it will be appreciated that neither the filter insert or the adjustable vale is necessarily required in the present invention.

Optimal performance of the goggle assembly includes ensuring the appropriate amount of heat load removal from goggle cavity for comfort and reduce chance of fogging, pressure control of the goggle cavity (to help prevent debris entry to cavity via main foam seal) and to ensure that in dry conditions the users' eyes do not dry out from too much fluid flow. In dry and cold conditions, the user may have a finer foam filter insert, or in the case of the adjustable valve have the flow adjusted to be low, to reduce the risk of dried out eyes and at the same time increasing goggle cavity pressure, resulting in less chance of debris ingress around the foam seal.

When elevated heat becomes a barrier to human performance, the inventor postulates that proper core body temperature regulation is possible as a result of the increased airflow and subsequent heat load removal from the exposed glabrous skin inside the goggle cavity.

The glabrous skin areas of the human body are specific in nature of providing areas of heat exchange for heat load removal to the atmosphere (e.g., similar to a radiator). The glabrous skin regions are located on the bottom of the feet, palms of the hands and the face around the eyes. These regions can release heat at a far greater rate than other areas of the body due to the flesh in these areas compromising of Arterio Venous Anastomoses (AVA's). It will be appreciated that, in many activities (e.g, motorcycle racing), the body may be completely covered and therefore to some extent fully insulated by personal protective equipment. To reduce the chance of hyperthermia, heat related exhaustion while also increase human performance potential, the present invention allows for the glabrous skin around the eyes to release heat at a much higher and consistent rate than a conventional goggle assembly. This heat load removal from critical body parts like the brain is facilitated by the flow of blood throughout the body. Adrenergic neurons within the central nervous and sympathetic nervous systems control dilation or constriction of the AVA's to meter the rate of blood flow to the glabrous skin to attempt to keep the body in a safe and optimal operating temperature range. An increase in blood flow as a result of dilation of the AVA's results in a greater headload removal from the blood and critical core body parts for a fixed airflow at a given ambient temperature over the glabrous skin.

The present invention allows for the difference in ambient temperature to goggle cavity temperature air to be resolved by consistent airflow. Ice packs and other methods of cooling the glabrous skin to allow for core body temperature recovery are ineffective when compared to airflow because the temperature difference is so great that the adregenic neurons cause the AVAs to constrict, and this slows the flow of blood in these areas. Ice packs and other cooling methods are also not passively operational in nature and generally cannot be performed while complete activities (such as racing motorcycles or similar).

Given that a typical athlete generates heat in proportion to metabolic load, and in the case of motorcycle racing speed is generally in proportion to metabolic load, the increasing requirement for the ability to release or regulate heat is met by the increasing rates of airflow through the goggle cavity.

In use, air flow is directed into the goggle cavity via the apertures located in an upper end of the goggle assembly. As the person is typically completing their task at speed, there is a positive pressure at the front of the helmet and this assists in directing the air into the goggle cavity. As the air enters the goggle cavity, the air/fluid within the goggle cavity is pushed through the duct and out the outlet thereof. This is further assisted by a lower pressure being present at the rear of the goggle assembly. In this regard, air or fluid in the goggle cavity is drawn out through the duct. That is, the lower pressure being present at the rear of the goggle assembly assists in sucking out or drawing out the air or fluid in the goggle cavity. Given that, at speed, a higher pressure is experienced in front of the goggle assembly than the rear of the goggle assembly then the present invention allows for active heat and moisture removal from the goggle cavity. The air flow may be suitably adjusted with an adjustable valve and/or the choice of filter media.

The present invention alleviates the issue of thermoregulation and thus fogging up. It will be appreciated that thermoregulation of the face or head region is a significant issue in the art as it represents a discomfort that may be addressed. Furthermore, the present invention allows the user to determine the amount of air flow through the goggle cavity to ensure comfort.

### Experimental Results

Testing was conducted for the purpose of determining the pressure differential from the present goggle assembly. The pressure differential shows the difference in pressure to induce the flow of air through the goggle cavity relative to the speed of the user. The pressure was measured in Pascals (Pa) with an instrument that has a range of + or −0 to 250 Pa.

In these tests, a rider was in the seated position with head positioned normally for duration of the test. The testing was completed with the rider, riding a motorcycle on flat ground in 34° C. ambient temperature 22 m above sea level.

The measured pressure are relative to atmospheric pressure and are measured in reference to a singular reference point. These are used as the differential point between any positive pressure or vacuum/negative pressure.

The pressure was measured the outlet of the duct (Table 1), and the apertures of the frame and/or lens pre filter insert (Table 2). Further pressure measurements were completed inside the goggle cavity (with the outlet blocked; Table 3) and inside the goggle cavity with one of the two outlet tubes unblocked; that is, one blocked and one unblocked; Table 4).

TABLE 1

| Speed (KPH) | High Pressure measured Pa | Low Pressure measured Pa |
|---|---|---|
| 15 | reference | −5 |
| 30 | reference | −20 |
| 45 | reference | −35 |
| 60 | reference | −85 |
| 75 | reference | −120 |
| 90 | reference | −180 |
| 105 | reference | −205 |

TABLE 2

| Speed (KPH) | High Pressure measured Pa | Low Pressure measured Pa |
|---|---|---|
| 15 | 0 | reference |
| 30 | +5 | reference |
| 45 | +10 | reference |
| 60 | +20 | reference |
| 75 | +50 | reference |
| 90 | +100 | reference |
| 105 | +150 | reference |

TABLE 3

| Speed (KPH) | High Pressure measured Pa | Low Pressure measured Pa |
|---|---|---|
| 15 | 0 | reference |
| 30 | +10 | reference |
| 45 | +30 | reference |
| 60 | +75 | reference |
| 75 | +95 | reference |
| 90 | +150 | reference |
| 105 | | |

TABLE 4

| Speed (KPH) | High Pressure measured Pa | Low Pressure measured Pa |
|---|---|---|
| 15 | 0 | reference |
| 30 | 0 | reference |
| 45 | +20 | reference |
| 60 | +70 | reference |
| 75 | +90 | reference |
| 90 | +130 | reference |
| 105 | | |

As can be seen from the above results, the use of the apertures in the frame and lens, and the duct results in a generous amount of airflow at all speed tested. It is postulated that the lower relative pressure imposes a significant amount of fluid flow through the goggle cavity. It is postulated that the volume of airflow through the goggle cavity is proportional to the speed of the ride.

The results suggest, for example, at 60 kph, a −85 Pa pressure is experienced on the outlet of the duct and a +20 Pa pressure is experienced at the apertures (pre filter). These results suggested that there is a 105 Pa differential across the goggle assembly and this results in a generous air volume and flow. It should be noted that in all tests, no fogging was experienced by the rider.

The data suggested that the use of the aperture and ducts allows for a net positive effect. In this regard, the advantages are shown to be at high speeds and low speeds.

For a comparison, the inventors completed similar tests on a goggle assembly that is similar to that tested in Tables 1-4. In this regard, the only difference is that an outlet tube is absent in the goggle assembly tested in Tables 5 and 6. Table 5 shows the pressure measured at a bottom side of the goggle assembly at the ventilation area, and Table 6 shows the pressure measured at the top side of the ventilation foam area.

TABLE 5

| Speed (KPH) | High Pressure measured Pa | Low Pressure measured Pa |
|---|---|---|
| 15 | 0 | reference |
| 30 | +5 | reference |
| 45 | +10 | reference |
| 60 | +30 | reference |
| 75 | +40 | reference |
| 90 | +50 | reference |
| 105 | +70 | reference |

TABLE 6

| Speed (KPH) | High Pressure measured Pa | Low Pressure measured Pa |
|---|---|---|
| 15 | +0 | reference |
| 30 | +5 | reference |
| 45 | +10 | reference |
| 60 | +20 | reference |
| 75 | +50 | reference |
| 90 | +100 | reference |
| 105 | +150 | reference |

The results from Table 5 and 6 appear to indicate that a much smaller pressure differential is shown in the goggle assembly without the duct. It appears, from the results, that very little airflow is observed. Furthermore, high speeds are required to achieve this little airflow.

Furthermore, the inventor postulates that for goggle assemblies, the results are not consistent between different goggles and helmet combinations with varying crosswind. The inventor postulates that the present invention achieves more consistent airflow because of the pressure differential observed.

### Standard Goggle Assembly Testing

The present invention was compared with a typical goggle assembly. At a speed of 75 KPH, a typical goggle assembly measured a 10 Pa difference whereas the present goggle assembly measured a 170 Pa difference.

At a speed of 45 KPH, a typical goggle assembly measured 0 Pa difference whereas the present goggle assembly measured a 45 Pa

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1. A goggle assembly comprising:
a frame supporting a lens, wherein the frame and/or lens comprise aperture(s) at an upper end thereof or therebetween;
a strap connected to frame; and
a duct connected to frame and/or lens at a side or bottom thereof, wherein the duct is adapted to vent air to the external environment.

2. The google assembly of claim 1, wherein the frame and/or lens comprise the aperture(s) at an upper end thereof.

3. The goggle assembly of claim 1, wherein the aperture is formed between the frame and lens.

4. The goggle assembly of claim 1, wherein the frame comprises the apertures at an upper end thereof.

5. The goggle assembly of claim 1, wherein the lens comprises the apertures at an upper end thereof.

6. The goggle assembly of claim 1, wherein the aperture(s) are in the form of a set of apertures.

7. The goggle assembly of claim 1, wherein the goggle assembly comprises a single set of apertures.

8. The goggle assembly of claim 1, wherein the goggle assembly further comprises a filter insert.

9. The goggle assembly of claim 1, wherein the aperture(s) is or are adapted to receive a filter insert.

10. The goggle assembly of claim 9, wherein the filter is adapted to filter the air and/or adapted to control fluid flow therethrough.

11. The goggle assembly of claim 1, wherein the duct comprises a passageway. In embodiments, the duct is formed with the strap.
